(19) United States
(12) Plant Patent Application Publication
(10) Pub. No.: US 2017/0049027 P1
(43) Pub. Date: Feb. 16, 2017

(54) DAHLIA PLANT NAMED 'FIDDAHTEMGOL'

(50) ;

(71) Applicant: Henk Dresselhuys, De Lier (NL)

(72) Inventor: Henk Dresselhuys, De Lier (NL)

(73) Assignee: FIDES B.V., De Lier (NL)

(21) Appl. No.: 14/756,179

(22) Filed: Aug. 12, 2015

(51) Int. Cl. A01H 5/00

(52) U.S. Cl. PLT/321

(57) ABSTRACT

A new and distinct cultivar of Dahlia plant named ‘Fiddahtemgol’, characterized by its relatively compact, upright, somewhat outwardly spreading and mounding plant habit; dark greyed green-colored leaves; early and freely flowering habit; medium to large inflorescences with orange-colored ray florets; and good postproduction longevity.

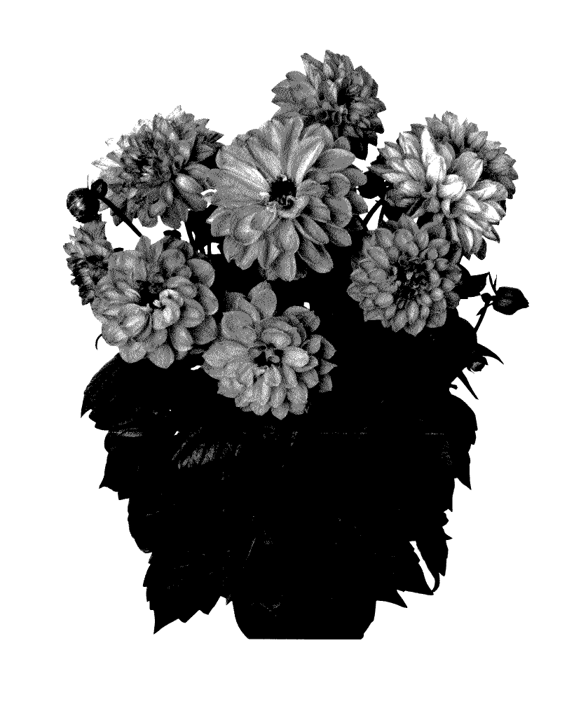

### BOTANICAL DESIGNATION

Dahlia variabilis

### CULTIVAR DENOMINATION

‘FIDDAHTEMGOL’

### BACKGROUND OF THE INVENTION

The present invention relates to a new and distinct cultivar of Dahlia plant, botanically known as Dahlia variabilis and hereinafter referred to by the name ‘Fiddahtemgol’.

The new Dahlia plant is a product of a planned breeding program conducted by the Inventor in De Lier, The Netherlands. The objective of the breeding program is to create new container Dahlia plants that have a freely branching and flowering habit, dark-colored leaves, attractive ray floret coloration and good postproduction longevity.

The new Dahlia plant originated from an open-pollination in De Lier, The Netherlands during the autumn of 2010 of an unidentified proprietary selection of Dahlia variabilis, not patented, as the female, or seed, parent with an unknown selection of Dahlia variabilis as the male, or pollen, parent. The new Dahlia plant was discovered and selected by the Inventor as a single flowering plant from within the progeny of the stated open-pollination in a controlled greenhouse environment in De Lier, The Netherlands during the spring of 2011.

Asexual reproduction of the new Dahlia plant by vegetative terminal cuttings in a controlled greenhouse environment in De Lier, The Netherlands since the autumn of 2011 has shown that the unique features of this new Dahlia plant are stable and reproduced true to type in successive generations.

### SUMMARY OF THE INVENTION

Plants of the new Dahlia have not been observed under all possible combinations of environmental conditions and cultural practices. The phenotype may vary somewhat with variations in environmental conditions such as temperature and light intensity, without, however, any variance in genotype.

The following traits have been repeatedly observed and are determined to be the unique characteristics of ‘Fiddahtemgol’. These characteristics in combination distinguish ‘Fiddahtemgol’ as a new and distinct Dahlia plant:
1. Relatively compact, upright, somewhat outwardly spreading and mounding plant habit.
2. Dark greyed green-colored leaves.
3. Early and freely flowering habit.
4. Medium to large inflorescences with orange-colored ray florets.
5. Good postproduction longevity. Compared to plants of the female parent selection, plants of the new Dahlia differ primarily in the following characteristics:
1. Plants of the new Dahlia are more compact than plants of the female parent selection.
2. Plants of the new Dahlia flower about five to seven days earlier than plants of the female parent selection.

Plants of the new Dahlia can be compared to plants of Dahlia ‘Fidahhypor’, not patented. In side-by-side comparisons conducted in De Lier, The Netherlands, plants of the new Dahlia differed primarily from plants of ‘Fidahhypor’ in the following characteristics:
1. Leaves of plants of the new Dahlia were dark greyed green in color whereas leaves of plants of ‘Fidahhypor’ were green in color.
2. Plants of the new Dahlia had semi-double-type inflorescences whereas plants of ‘Fidahhypor’ had fully double-type inflorescences.
3. Plants of the new Dahlia and ‘Fidahhypor’ differed in ray floret color as plants of ‘Fidahhypor’ had darker orange-colored ray florets.

Plants of the new Dahlia can also be compared to plants of Dahlia ‘Cultivaris Dreamy Moonlight’, not patented. In side-by-side comparisons conducted in De Lier, The Netherlands, plants of the new Dahlia differed primarily from plants of ‘Cultivaris Dreamy Moonlight’ in the following characteristics:
1. Plants of the new Dahlia were more compact and denser than plants of ‘Cultivaris Dreamy Moonlight’.
2. Leaves of plants of the new Dahlia were darker greyed green in color than leaves of plants of ‘Cultivaris Dreamy Moonlight’.
3. Plants of the new Dahlia and ‘Cultivaris Dreamy Moonlight’ differed in ray floret color as plants of ‘Cultivaris Dreamy Moonlight’ had yellow-colored ray florets.

### BRIEF DESCRIPTION OF THE PHOTOGRAPH

The accompanying photograph illustrates the overall appearance of the new Dahlia plant showing the colors as true as it is reasonably possible to obtain in colored reproductions of this type. Colors in the photograph may differ slightly from the color values cited in the detailed botanical description which accurately describe the colors of the new Dahlia plant. The photograph is a side perspective view of a typical flowering plant of ‘Fiddahtemgol’ grown in a container.

### DETAILED BOTANICAL DESCRIPTION

The aforementioned photograph and the following observations and measurements describe plants grown during the summer in 15-cm containers in a glass-covered greenhouse in De Lier, The Netherlands and under environmental conditions and cultural practices which approximate those generally used in commercial potted Dahlia production. During the production of the plants, day temperatures ranged from 18° C. to 20° C., night temperatures averaged 18° C. and light levels averaged 4,500 lux. Plants were pinched two weeks after planing and were ten weeks old when the photographs and description were taken. In the following description, color references are made to The Royal Horticultural Society Colour Chart, 2007 Edition, except where general terms of ordinary dictionary significance are used.
Botanical classification: Dahlia variabilis ‘Fiddahtemgol’.
Parentage:
   Female, or seed, parent.—Unidentified proprietary selection of Dahlia variabilis, not patented.
   Male, or pollen, parent.—Unknown selection of Dahlia variabilis, not patented.
Propagation:
   Type.—By vegetative terminal cuttings.
   Time to initiate roots, summer.—About nine to ten days at temperatures about 20° C. to 25° C.
   Time to initiate roots, winter.—About 11 to 13 days at temperatures about 18° C. to 20° C.
   Time to produce a rooted plant, summer.—About 2 to 2.5 weeks at temperatures about 20° C. to 25° C.
   Time to produce a rooted plant, winter.—About 2.5 to 3 weeks at temperatures about 18° C. to 20° C.
   Root description.—Fine to medium in thickness, fleshy; typically white in color, actual color of the roots is dependent on substrate composition, water quality, fertilizers, substrate temperature and age of roots; tuber development has not been observed on plants of the new Dahlia.
   Rooting habit.—Freely branching; medium density.
Plant description:
   Plant and growth habit.—Relatively compact, upright, somewhat outwardly spreading and mounding plant form; broad inverted triangle; moderately freely basal branching with about four primary lateral branches developing per plant, each primary lateral branch with numerous secondary branches; inflorescences held above the foliar plane on strong peduncles; bushy and dense habit; moderately vigorous growth habit.
   Plant height.—About 30 cm.
   Plant diameter or spread.—About 27 cm.
   Lateral branches.—Length: About 26 cm to 28 cm. Diameter: About 5 mm to 6 mm. Internode length: About 3.5 cm to 4 cm. Aspect: Erect to somewhat outwardly spreading. Strength: Moderately strong. Texture: Smooth, glabrous. Luster: Slightly glossy. Color: Close to 146C.
Leaf description:
   Arrangement.—Opposite; leaves may be single or compound with three or occasionally five leaflets.
   Length.—About 10 cm to 11 cm.
   Width.—About 6 cm to 7 cm.
   Shape.—Ovate.
   Apex.—Acute.
   Base.—Attenuate.
   Margin.—Serrate to dentate.
   Venation pattern.—Pinnate.
   Texture, upper and lower surfaces.—Slightly pubescent; slightly rough.
   Luster, upper and lower surfaces.—Slightly glossy.
   Color.—Developing leaves, upper surface: Close to N137A. Developing leaves, lower surface: Close to N137C. Fully expanded leaves, upper surface: Close to N189A; venation, close to 79A. Fully expanded leaves, lower surface: Close to N137A; venation, close to 137B.
   Petioles.—Length: About 4 cm. Diameter: About 4 mm. Strength: Strong. Texture, upper and lower surfaces: Smooth, glabrous. Luster, upper and lower surfaces: Slightly glossy. Color, upper surface: Close to N79A. Color, lower surface: Close to N137B.
Inflorescence description:
   Appearance and arrangement.—Semi-double inflorescence form with ray florets forming acropetally on a receptacle; inflorescences positioned above the foliar plane on strong peduncles; inflorescences face mostly upright; freely flowering habit with about 20 to 30 inflorescences developing per plant.
   Fragrance.—None detected.
   Time to flower.—Plants flower continuously from late spring through the autumn in The Netherlands; early flowering habit, plants begin flowering about 7.5 to 8.5 weeks after planting.
   Post-production longevity.—Inflorescences maintain good substance for about two weeks on the plant; inflorescences persistent.
   Inflorescence buds.—Height: About 1.2 cm. Diameter: About 1.5 cm. Shape: Globular, flattened. Color: Close to 144B and N79C.
   Inflorescence size.—Diameter: About 10 cm to 12 cm. Depth (height): About 6 cm to 8 cm. Disc diameter: About 1.8 cm. Receptacle height: About 5 mm. Receptacle diameter: About 1.5 cm.
   Ray florets.—Quantity per inflorescence: About 40 to 50 arranged in about five to six whorls. Length: About 4 cm. Width: About 2.2 cm. Shape: Ovate. Apex: Obtuse. Base: Attenuate. Margin: Entire. Aspect: Initially upright to roughly perpendicular to the peduncle; somewhat concave. Texture, upper and lower surfaces: Smooth, glabrous. Luster, upper and lower surfaces: Shiny. Color: When opening, upper surface: Close to 23A. When opening, lower surface: Close to 21C. Fully opened, upper surface: Close to 17B; towards the margins, heavily tinged with close to 43A; venation, close to 17B; color becoming closer to 13B with development. Fully opened, lower surface: Close to 17D; venation, close to N81D, color becoming closer to 13C with development.
   Disc florets.—Quantity per inflorescence: About 50 to 60. Length: About 9 mm. Diameter: About 1.8 mm. Shape: Tubular, elongated; apices obtuse to dentate. Texture: Smooth, glabrous. Luster: Shiny. Color, when opening: Apex: Close to N25A becoming closer to 60A. Mid-section: Close to 60A. Base: Close to 1D. Color, fully opened: Apex and mid-section: Close to N77A. Base: Close to 1D.
   Phyllaries.—Quantity per inflorescence: About seven arranged in a single whorl. Length: About 1.5 cm. Width: About 6 mm. Shape: Ovate. Apex: Acute. Base: Attenuate. Margin: Entire. Texture, upper and lower surfaces: Smooth, glabrous. Luster, upper and lower surfaces: Shiny. Color, upper surface: Close to 137A; venation, close to 79A. Color, lower surface: Close to 136B, venation, close to 79A.
   Peduncles.—Length, terminal peduncle: About 11 cm to 12.55 cm. Length, fourth peduncle: About 11 cm. Diameter: About 4 mm. Aspect: Mostly erect to about 45° from stem axis. Strength: Strong. Texture: Smooth, glabrous. Luster: Slightly shiny. Color: Close to 144A and 79B; proximally, close to 79A.
   Reproductive organs.—Androecium: Present on disc florets only. Quantity per floret: One. Filament length: About 4 mm. Filament color: Close to 17A. Anther shape: Lanceolate. Anther length: About 4 mm. Anther color: Close to 15A. Pollen amount: Abundant. Pollen color: Close to 21A. Gynoecium: Present on ray and disc florets. Quantity per floret: One. Pistil length: About 1.7 mm. Style length: About 1 mm to 1.1 mm. Style color: Close to 2A; towards the base, close to 145B. Stigma color: Close to 7A. Ovary color: Close to 145C. Seeds: Length: About 7.5 mm. Diameter: About 4.5 mm. Texture: Smooth, glabrous. Color: Close to 187A to 187B.
Disease & pest resistance: Plants of the new Dahlia have not been observed to be resistant to pathogens and pests common to Dahlia plants.
Temperature tolerance: Plants of the new Dahlia tolerate high temperatures about 35° C. and short periods of low temperatures about 5° to 10° C.

It is claimed:

1. A new and distinct Dahlia plant named ‘Fiddahtemgol’ as illustrated and described.
